(19) United States
(12) Patent Application Publication
(10) Pub. No.: US 2006/0063733 A1
(43) Pub. Date: Mar. 23, 2006

(54) Methods of reducing an immune response

(76) Inventor: Quiming Chu, Belmont, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/263,672

(22) Filed: Oct. 31, 2005

(30) Foreign Application Priority Data
May 5, 2004 (WO) PCT/US04/14137

(51) Int. Cl. A61K 48/00

(52) U.S. Cl. 514/44

(57) ABSTRACT

The invention relates to methods of reducing an immune response to a transgene product in a mammal by co-administration of a small-interfering ribonucleic acid (siRNA) molecule that temporarily inhibits or reduces transgene expression, wherein the siRNA is administered in an amount, and for a period of time, sufficient to reduce an immune response to the transgene product when it is expressed at therapeutic levels. The present invention further relates to methods of administering siRNAs to a mammal to reduce an immune response to an immunogenic protein, such as an enzyme used in enzyme replacement therapy.

### REFERENCE TO RELATED APPLICATIONS

This application claims priority to PCT Application No. PCT/US2004/014137, filed 5 May 2004, which claims priority to U.S. Provisional Patent Application Ser. No. 60/468,229 filed May 5, 2003 and U.S. Provisional Patent Application Ser. No. 60/476,216 filed Jun. 4, 2003, the text of which is incorporated herein by reference.

### FIELD OF THE INVENTION

The invention relates to methods of reducing an immune response to a transgene product in a mammal by co-administration of a small-interfering ribonucleic acid (siRNA) molecule that temporarily inhibits or reduces transgene expression, wherein the siRNA is administered in an amount, and for a period of time, sufficient to reduce an immune response to the transgene product when it is expressed at therapeutic levels. The present invention further relates to methods of administering siRNAs to a mammal to reduce an immune response to an immunogenic protein, such as an enzyme used in enzyme replacement therapy.

### BACKGROUND OF THE INVENTION

Development of a neutralizing antibody to a therapeutic product has been recognized as a significant problem in gene therapy and enzyme infusion therapies, such as those used to treat lysosomal storage diseases. The development of a humoral response (i.e., the production of neutralizing antibodies), in particular, has been shown to reduce therapeutic efficacy of gene therapy or replacement enzyme treatment. Nishikawa et al., Biol. Pharm. Bull., 25(3): 275-283 (2002); Rosenberg et al., Blood, 93 (6): 2081-2088 (1999).

The magnitude of the neutralizing antibody response to a transgene product depends on a number of factors, such as the amount of the transgene expressed, whether the transgene product is recognized as foreign, whether the transgene product is secreted, and the type of cells in which the transgene is expressed. Vectors with tissue- or cell-specific promoters limit expression to non-antigen presenting cells and are less likely to result in a development of antibody response. Wang et al., Mol. Therapy, 1(2): 154-158 (2000).

Further, the antibody response depends on the type of the vector and method of delivery. For example, hydrodynamic delivery is a method of choice for transfecting the liver because it results in high levels of expression of the transgene product. However, this high initial level of expression typically results in generation of a neutralizing antibody response. Additionally, this procedure causes injury to the liver, and as a result, inflammation. The inflammatory response in turn exacerbates the antibody response to the transgene product.

Efforts to circumvent the problem of inducing an immune response to the therapeutic product include: (1) co-administration of an immunosuppressant, e.g., FK506, cyclophosphamide, deoxyspergualin, MR1 (anti-CD40 ligand), and CTLA4-lg; (2) use of drug-inducible promoters; and (3) use of tissue-specific promoters. Unfortunately, each of these efforts is associated with another set of problems.

For example, it has been reported that suppression of an immune response may be achieved, for example, by treating the host organism such as a mammal with drugs to suppress the immune system. Jooss et al., Human Gene Therapy, 7: 1555-1566 (1996); Yang et al., J. Virol., 70: 6370-6377 (1996); Ziegler et al., Human Gene Therapy, 10: 1667-1682 (1999). Similarly, studies directed to regulating expression of a transgene using a drug inducible promoter such as a promoter regulated by tetracycline or rapamycin have been undertaken in an effort to reduce the immune response to the transgene product. Rendahl et al., Nature Biotechnology, 16: 757-761 (1998); and Ye et al., Science, 283: 88-91 (1999). However, immunosuppressants and drugs used to control inducible promoters have significant deleterious side effects on the patient. Moreover, it appears that inducible promoters do not actually solve the immune response problem but rather just delay its onset. Abruzzese et al., Human Gene Therapy, 10: 1499-1507 (1999).

Tissue specific promoters have been used to restrict the expression of the transgene to non-immune cells in an effort to avoid or reduce the development of neutralizing antibodies. Wang et al., supra. However, these promoters provide no ability to regulate expression and, thus, may still result in expression levels high enough to generate a neutralizing antibody response or alternatively, may result in expression levels too low to provide therapeutic levels of the transgene product.

### SUMMARY OF THE INVENTION

The invention provides methods for reducing an immune response to a product of a transgene in a mammal, especially a human, comprising administering to the mammal a vector comprising a transgene encoding a product that is immunogenic in the mammal and simultaneously or sequentially administering a siRNA that temporarily inhibits transgene expression. In the methods of the invention, the siRNA is administered in an amount and for a period of time sufficient to reduce any immune response to the immunogenic transgene product when it is expressed at a therapeutic level.

The invention further provides methods for treating or preventing a disease state in a patient, comprising administering to the patient a vector comprising a transgene encoding an immunogenic product that treats or prevents the disease state and simultaneously or sequentially administering a siRNA that temporarily inhibits expression of the transgene. The siRNA is administered in an amount and for a period of time sufficient to reduce any immune response to the product when expressed at a therapeutic level.

Another aspect of the invention provides methods for treating a lysosomal storage disease in a mammal comprising administering a vector comprising a transgene encoding an enzyme which is deficient in the mammal with the lysosomal storage disease and simultaneously or sequentially administering a siRNA that temporarily inhibits expression of the transgene encoding the enzyme. The siRNA is administered in an amount and for a period of time sufficient to reduce an immune response to the enzyme when it is expressed at therapeutic levels.

The invention further provides methods for reducing an immune response in a mammal to an immunogenic product comprising administering to the mammal a vector comprising a transgene encoding the immunogenic product and simultaneously or sequentially administering a siRNA that temporarily inhibits transgene expression, wherein the siRNA is administered in an amount and for a period of time sufficient to reduce an immune response to the immunogenic product. This method is particularly useful in the treatment of a disease caused by deficiency of an enzyme, such as a lysosomal hydrolase wherein administration of replacement enzyme would otherwise invoke an immune response.

### BRIEF DESCRIPTION OF THE FIGURES

FIG. 1 is a schematic representation of a predicted secondary structure of the human α-galactosidase mRNA with the free energy of −341 kcal/mol. The rectangle indicates the loop used for the design of aGAL-siRNA-3 (also referred to as siRNA-3).

FIG. 2 is a schematic representation of the pGZDC190-sHAGA plasmid comprising the transgene that encodes human α-galactosidase.

FIG. 3 is a schematic representation of the pGZB-sHAGA plasmid comprising the transgene that encodes human α-galactosidase.

FIG. 4 depicts plasma levels of human α-galactosidase from BALB/c mice at days 1, 8, 14, 26, 56, and 84 post injection. Mice were injected with one of the following: (1) 10 μg of pGZBDC190-sHAGA; (2) 10 μg of pGZBDC190-sHAGA and 10 μg of CAT-siRNA; or (3) 10 μg of pGZBDC190-sHAGA and 10 μg of aGAL-siRNA-3.

FIG. 5 depicts plasma levels of human α-galactosidase and anti-α-galactosidase titers for BALB/c mice at days 84, 99, and 112 post injection. Mice were injected with one of the following: (1) 4 μg of pGZB-sHAGA, referred to as pGZB-shaGal; (2) 4 μg of pGZB-sHAGA plus 5 μg of CAT-siRNA; (3) 4 μg of pGZB-sHAGA plus 5 μg of aGAL-siRNA-3, referred to as siRNA-3; or (4) 4 μg of the pGZB-shaGAL plasmid plus 0.5 μg of aGAL-siRNA-3.

FIG. 6 depicts levels of expression of the human α-galactosidase protein in the liver, spleen, heart, lungs, and kidneys of Fabry mice. Mice were hydrodynamically injected with 10 μg of the pGZCUBIHAGA plasmid and the measurements were taken at days 1, 14, 28, and 42 post injection.

FIG. 7 depicts levels of globotriaosylceramide (GL-3) in the liver, spleen, heart, lungs, and kidneys of Fabry mice at days 1, 14, 28, and 42 following hydrodynamic injection with 10 μg of the pGZCUBIHAGA plasmid. The results are expressed as percentages relative to untreated (naïve) mice.

FIG. 8 depicts anti-α-galactosidase antibody titers in the plasma of Fabry mice at days 1, 14, 28, and 42, following hydrodynamic injection with 10 μg of pGZCUBIHAGA.

FIG. 9A depicts plasma levels of human α-galactosidase in Fabry Fabry mice at days 1, 7, 14, 21, and 42 following hydrodynamic injection with one of the following: (1) 10 μg of pGZB-sHAGA; (2) 10 μg of pGZB-sHAGA plus 10 μg of aGAL-siRNA-3; or (3) 10 μg of pGZB-sHAGA plus 10 μg of CAT-siRNA.

FIG. 9B depicts distributions of anti-α-galactosidase antibody titers among Fabry mice at day 42, following hydrodynamic injection with one of the following: (1) 10 μg of pGZB-sHAGA; (2) 10 μg of pGZB-sHAGA plus 10 μg of aGAL-siRNA-3; or (3) 10 μg of pGZB-sHAGA plus 10 μg of CAT-siRNA.

FIG. 10A depicts plasma levels of human α-galactosidase in Fabry mice at days 1, 7, and 98 following hydrodynamic injection with one of the following: (1) 10 μg of pGZBDC190-shAGAL; (2) 10 μg of pGZBDC190-shAGAL plus 10 μg of aGAL-siRNA-3; or (3) 10 μg of pGZBDC190-shAGAL plus 10 μg of CAT-siRNA.

FIG. 10B depicts distributions of anti-α-galactosidase antibody titers among Fabry mice at day 98 following hydrodynamic injection with one of the following: (1)10 μg of pGZDC190-shAGAL; (2) 10 μg of pGZDC190-shAGAL plus 10 μg of aGAL-siRNA-3; or (3) 10 μg of pGZDC190-shAGAL plus 10 μg of CAT-siRNA.

FIG. 11A depicts plasma levels of human α-galactosidase in Fabry mice overtime following hydrodynamic injection with one of the following: (1) 10 μg of pGZDC190-shAGAL, referred to as pDC190-agal; (2) 10 μg of pGZDC190-shAGAL plus 10 μg of aGAL-siRNA-3, referred to as siRNA-3; or (3) 10 μg of pGZDC190-shAGAL plus 10 μg of CAT-siRNA.

FIG. 11B depicts anti-α-galactosidase antibody titers in Fabry mice at 16 weeks (W16) and 19 weeks (W19) after the following treatment protocols: (1) no plasmid at week 0/Fabrazyme in Complete Freund's Adjuvant (CFA) at week 16 [Naive+Fab]; (2) hydrodynamic injection of 10 μg of pGZBDC190-shAGAL at week 0/Fabrazyme in CFA at week 16 [pDC190+Fab]; or (3) hydrodynamic injection of 10 μg of pGZBDC-1 90-shAGAL plus 10 μg of aGAL-siRNA-3 at week 0/Fabrazyme in CFA at week 16 [pDC190/aGal-siRNA+Fab].

FIG. 11C depicts anti-α-galactosidase antibody titers in Fabry mice at 16 and 19 weeks after the following treatment protocols: (1) no plasmid at week 0/Complete Freund's Adjuvant (CFA) at week 16 [Naive+CFA]; (2) hydrodynamic injection of 10 μg of pGZB-sSEAP at week 0/Fabrazyme in CFA at week 16 [pGZB-sSEAP+Fab]; or (3) hydrodynamic injection of 10 μg of pGZB-sSEAP/CFA at week 16 [pGZB-sSEAP+CFA].

### DETAILED DESCRIPTION OF THE INVENTION

In order that the present invention may be more readily understood, certain terms are first defined. Additional definitions are set forth throughout the detailed description.

The term “hydrodynamic injection” refers to an intravascular injection at a rate and volume sufficient to generate supra-systemic pressure within the vascular space and/or the subtending organ parenchyma. Methods of hydrodynamic delivery are described in U.S. Pat. No. 6,265,387.

The terms “inhibit” and “neutralize” and their cognates refer to the ability of a compound to reduce biological activity of another compound or to interfere with a certain reaction resulting in a reduction of an amount of or biological activity of another compound. The term “biological activity” refers to a function or set of functions, or the effect to which the function is attributed, performed by a molecule in a biological system, which may be in vivo or in vitro. Inhibition can be measured using methods known in the art or as described in the Examples.

The term “inhibition” used in connection with transgene expression refers to an observable decrease or absence in the level of protein and/or mRNA product expressed by the transgene. In the methods of this invention, transgene expression may be completely or partially inhibited at the mRNA or protein level. Alternatively, transgene expression may be reduced only enough to attenuate an immune response to the transgene product.

The term “immunogenic product” refers to any large molecule whose entry into a host provokes an immune response in the host, e.g., synthesis of antibody specific to the immunogenic product.

The term “immune response” refers to a reaction of the immune system to an antigen in the body of a host, which includes generation of an antigen-specific antibody and/or cellular cytotoxic response. The term further refers to a response the immune system that leads to a condition of induced sensitivity to an immunogenic product. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by a transgene. The level of the immune response can be measured by methods known in the art or as described in the Examples (e.g., by measuring antibody titer).

The term “reduction” used in connection with the level of an immune response following administration of a transgene refers to an observable difference in the levels of immune response between two or more hosts, at least one of which receives a siRNA in addition to the transgene. A statistically significant difference between the levels of immune responses can be determined by any appropriate method known in the art. See, for example, Steel et al., Principles and Procedures of Statistics, A Biometrical Approach (McGraw-Hill, 1980). The term “inhibition” also refers to prevention or a delay of onset of an immune response.

The term “lysosomal storage disease” refers to disorders associated with a deficiency in a lysosomal hydrolase or a protein involved in the lysosomal trafficking. Representative lysosomal diseases and defective enzymes involved are listed in Table 1.

TABLE 1

| Lysosomal storage disease | Defective enzyme |
|---|---|
| Aspartylglucosaminuria | Aspartylglucosaminidase |
| Fabry | α-Galactosidase A |
| Batten (CNL1-CNL8) | Multiple gene products |
| Cystinosis | Cysteine transporter |
| Farber | Acid ceramidase |
| Fucosidosis | Acid α-L-fucosidase |
| Galactosidosialidosis | Protective protein/cathepsin A |
| Gaucher types 1, 2, and 3 | Acid β-glucosidase, or glucocerebrosidase |
| G_{M1} gangliosidosis | Acid β-galactosidase |
| Hunter | Iduronate-2-sulfatase |
| Hurler-Scheie | α-L-Iduronidase |
| Krabbe | Galactocerebrosidase |
| α-Mannosidosis | Acid α-mannosidase |
| β-Mannosidosis | Acid β-mannosidase |
| Maroteaux-Lamy | Arylsulfatase B |
| Metachromatic | Arylsulfatase A |
| leukodystrophy | |
| Morquio A | N-Acetylgalactosamine-6-sulfate sulfatase |
| Morquio B | Acid β-galactosidase |
| Mucolipidosis II/III | N-Acetylglucosamine-1-phosphotransferase |
| Niemann-Pick A, B | Acid sphingomyelinase |
| Niemann-Pick C | NPC-1 |
| Pompe | Acid α-glucosidase |
| Sandhoff | β-Hexosaminidase B |
| Sanfilippo A | Heparan N-sulfatase |
| Sanfilippo B | α-N-Acetylglucosaminidase |
| Sanfilippo C | Acetyl-CoA: α-glucosaminide |
| | N-acetyltransferase |
| Sanfilippo D | N-Acetylglucosamine-6-sulfate sulfatase |
| Schindler Disease | α-N-Acetylgalactosaminidase |
| Schindler-Kanzaki | α-N-Acetylgalactosaminidase |
| Sialidosis | α-Neuramidase |
| Sly | β-Glucuronidase |
| Tay-Sachs | β-Hexosaminidase A |
| Wolman | Acid Lipase |

The term “therapeutic level” refers to the amount of a transgene product or the level of activity of a transgene product sufficient to confer its therapeutic or beneficial effect(s) in the host receiving the transgene. Expression levels of the transgene or the levels of activity of the transgene product can be measured at the protein or the mRNA level using methods known in the art or as described in the Examples.

The term “transgene” refers to a polynucleotide that is introduced into the cells of a tissue or an organ and is capable of being expressed under appropriate conditions, or otherwise conferring a beneficial property to the cells. A transgene is selected based upon a desired therapeutic outcome. It may encode, for example, hormones, enzymes, receptors, or other proteins of interest.

The term “transgene product” refers to any molecule that is encoded by a transgene and confers a beneficial property to the cells or a desired therapeutic outcome. The term includes but is not limited to RNA transcripts, e.g., mRNA, and proteins.

Compositions and Methods

In the experiments leading to this invention, normal mice and α-galatosidase-deficient mice (a model of Fabry disease) were administered a plasmid DNA comprising the α-galactosidase transgene by hydrodynamic injection. The invention is based, in part, on discovery and demonstration that co-administration of the vector and transgene-specific siRNA reversibly suppresses initial supratherapeutic expression of α-galactosidase. The present invention is further based, in part, on discovery and demonstration that siRNA-mediated suppression diminishes the neutralizing host's immune response to α-galactosidase, while allowing the transgene to be expressed at therapeutic levels once the siRNA effect is reversed.

The invention provides methods of reducing an immune response to a transgene product by co-administration of a transgene and a transgene-specific siRNA. The methods of the invention comprise administering the transgene-specific siRNA to a mammal in an amount and for a period of time sufficient to reduce the initial supratherapeutic expression of the transgene and subsequently allowing to the transgene to be expressed in a therapeutic amount, wherein the transgene-specific immune response is reduced when the transgene is expressed at a therapeutic level. Alternatively, the methods of the invention may be used to induce immunologic tolerance in a mammal to an otherwise immunogenic product that is then administered by another route or re-administered by gene therapy.

siRNAs are usually 21-23 nucleotides long (but may be longer or shorter) and lead to post-transcriptional silencing of the mRNA to which they are homologous. RNA interference or RNAi is a method based on small-interfering RNAs that can lead to the silencing of specific genes. It has been shown that RNAi is mediated by RNA-induced silencing complex or RISC, which is a sequence specific, multi-component nuclease that destroys mRNAs and contains short RNAs. Complementary portions of siRNA that hybridize to form the double-stranded structure typically have substantial or complete identity. The double-stranded structure may be formed by a single self-complementary RNA strand or two complementary RNA strands. siRNAs of the invention may comprise one or more strands of polymerized ribonucleotide and may include modifications to either the phosphate-sugar backbone or the nucleoside. Likewise, bases may be modified to block the activity of enzyme adenosine deaminase, an enzyme that plays a role in RNA-editing. RNA duplex formation can be initiated either before or after administration into a host organism or cell for effective inhibition of or reduction in the expression of the target gene. In one aspect, the target gene is a transgene. In a preferred embodiment, the transgene is carried by a gene therapy vector.

In the methods of the invention, siRNAs are complementary to certain portions of a particular mRNA, e.g., a target gene or a transgene. siRNA has the ability to inhibit expression when the siRNA is administered to the same cell or the same host organism as the transgene.

The sequence of a siRNA of the invention can correspond to the entire length of a transgene, or only a portion of the transgene. In one embodiment, the length of the siRNA, i.e., the length of each individual strand of the double-stranded structure, as well as the length of the duplex, comprises 15, 16, 17, 18, 19, 20, 21, 22, 23, 24, or 25 nucleotides. In another embodiment, the length of the siRNA is about 25-50 nucleotides. In yet another embodiment, the length of the siRNA is greater than 50 nucleotides.

As used herein, a “gene therapy vector” refers to a nucleic acid molecule capable of transporting another nucleic acid molecule to which it has been linked, for use in gene therapy. In one embodiment according to the invention, the gene therapy vector is used to administer a transgene which encodes a therapeutic product. “Vectors” as used herein, include, but are not limited to plasmids, phagemids, and viruses. Examples of various vectors that can be used for delivery of a transgene to an immune competent host organism include, for example, adenovirus vectors, adeno-associated virus (AAV) vectors, cytomegaly virus (CMV) vectors, herpes virus vectors and retroviral vectors. It is understood that gene therapy vectors, as used herein, include presently known gene therapy vectors as well as future modifications and variations of commonly used gene therapy vectors, and new vectors developed in the future for transporting a transgene into a host organism for gene therapy, including vectors used for ex vivo gene therapy applications.

A transgene can be expressed in a cell or an organism to which a vector comprising the transgene is administered, and where the expression of transgene is prophylactically or therapeutically beneficial to the cell, tissue, organ, organ system, organism, or cell culture of which the cell is a part. In one embodiment, the transgene confers its prophylactic or therapeutic effect in a mammal, for example, a human. The transgene can exert its effect at the level of mRNA or protein. Typically, the transgene encodes an immunogenic product, e.g., a protein.

In the methods of this invention, a gene therapy vector comprising the transgene is administered to a mammal along with a siRNA that can inhibit or reduce expression of the transgene in order to attenuate any immune response to the protein encoded by the transgene. The vector comprising a transgene can be administered to the host organism simultaneously with a siRNA that is capable of temporarily reducing or inhibiting expression of the transgene. Alternatively, the vector comprising a transgene can also be administered sequentially with a siRNA that is capable of temporarily reducing or inhibiting expression of the transgene. In one embodiment, the siRNA is administered to an immune competent host organism before administration of the gene therapy vector comprising the transgene. In another embodiment, the siRNA is administered to an immune competent host organism after administration of the gene therapy vector comprising the transgene.

The siRNA may be administered in an amount which allows delivery of at least one copy of siRNA per cell that contains the gene therapy vector, for example in cell culture or a host organism. Higher doses of the siRNA, for example, at least 5, 10, 100, 500 or 1000 copies of siRNA per cell, administered to a single cell such as a cell in culture, or a cell in a tissue, organ, organ system or a whole organism, where the cell contains one or more copies of the transgene, may yield more effective inhibition of transgene expression, relative to inhibition achieved with a lower copy number of the siRNA per cell.

In accordance with one embodiment of the invention, consequences of reduction in or inhibition of transgene expression can be confirmed by examination of the outward properties of the host organism, for example, alleviation of symptoms of an immune response to the transgene protein product or absence of an immune response to the transgene product that would otherwise be seen in absence of the siRNA to inhibit transgene expression. Biochemical techniques to detect the mRNA or protein product of the transgene can also be used, such as RNA solution hybridization, nuclease protection, Northern blot hybridization, reverse transcription, gene expression monitoring with a microarray, antibody binding, enzyme linked immunosorbent assay (ELISA), Western blotting, radioimmunoassay (RIA), other immunoassays, immunofluorescence, and fluorescence activated cell analysis (FACS).

Inhibition of transgene expression can also be measured in a cell in culture to which a vector comprising the transgene and a corresponding siRNA has been administered. Accordingly, in one embodiment, a decrease in transgene expression conferred by a siRNA is first assayed in a cell, for example, in vitro in cell culture, before such a siRNA is administered in vivo to an immune competent host organism.

Specificity of a siRNA for the transgene is reflected by the ability of the siRNA to inhibit transgene expression without manifest effects on other genes of the cell. Gene expression in a cell line or a whole organism can be monitored by use of a reporter or drug resistance gene. For example, reporter genes can be linked to the transgene whose expression is desired to be inhibited. Reporter genes that may be used in accordance with the methods of the invention include, but are not limited to, for example, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are also available for assaying transgene expression. Such markers typically confer resistance to one or more drugs, for example, ampicillin, bleomycin, chloramphenicol, gentamycin, hygromycin, kanamycin, lincomycin, methotrexate, phosphinothricin, puromycin, and tetracycline. These drug resistance markers can be used for assaying transgene expression or inhibition of transgene expression following administration of siRNA to a host organism or to a cell line.

The degree of inhibition of transgene expression upon administration of a siRNA to a host organism or host cell can be quantitated in vitro or in vivo by one or more assays described herein. Inhibition of transgene expression can be assayed both at the mRNA as well as the protein level. For example, a degree of inhibition of transgene expression at the mRNA or at the protein level can be at least 2, 5, 10, 15, 20, 25, 30, 40, 50, 55, 60, 65, 70, 75, 80, 85, 90, 92, 95, 97, 98, or 99% relative to transgene expression in absence of the siRNA. In one embodiment, the degree of inhibition of transgene expression correlates with an immune response against the protein encoded by the transgene. Therefore, a higher degree of inhibition of transgene expression is expected to result in a more reduced immune response against the protein encoded by the transgene. The degree of inhibition of transgene expression will depend, in part, for example, on the half-life of the mRNA for the transgene, half-life of the protein for the transgene, the dosage and amount of the siRNA used for inhibition and the length of time for which the siRNA is administered. The dosage and amount of the siRNA may, in turn, depend on the in vivo half-life of the siRNA and the molar ratio of siRNA to the transgene. A length of time for which the transgene expression is reduced or inhibited by a siRNA may be at least 3, 4, 5, 6, 7, 8, 9, 10, 11, 12, 13, 14, 15, 16, 17, 18, 19, or 20 days from the time the transgene is administered to a mammal.

The efficiency of inhibition of transgene expression may be determined by assessing the amount of gene product in the cell. For example, mRNA may be detected with a hybridization probe having a nucleotide sequence outside the region used for the siRNA, or translated polypeptide may be detected with an antibody.

siRNAs containing a nucleotide sequence identical to a portion of a transgene are preferred for inhibition of transgene expression, thereby resulting in attenuation in an immune response to an immunogenic protein encoded by the transgene in a host organism. However, RNA sequences with insertions, deletions, and single point mutations relative to the transgene or target gene sequence have also been found to be effective for inhibition. Thus, the invention has the advantage of being able to tolerate sequence variations that might be expected due to genetic mutation, strain polymorphism, or evolutionary divergence.

Sequence identity between a siRNA and the transgene whose expression is desired to be inhibited may be optimized by sequence comparison and alignment algorithms known in the art (see Gribskov and Devereux, Sequence Analysis Primer, Stockton Press, 1991). The percent difference between the nucleotide sequences by can be calculated by, for example, the Smith-Waterman algorithm as implemented in the BESTFIT software program using default parameters (see University of Wisconsin Genetic Computing Group). Greater than about 90% sequence identity, or 100% sequence identity, between the siRNA and the portion of the transgene is generally desirable. However, complete sequence identity between the siRNA and the transgene is not required to practice the present invention. Alternatively, the duplex region of a siRNA may be defined as a nucleotide sequence that is capable of hybridizing with a portion of the target gene or transgene transcript, for example, under stringent hybridization conditions (e.g., 400 mM NaCl, 40 mM PIPES pH 6.4, 1 mM EDTA, and 50° C. or 70° C. hybridization for 12-16 hours; followed by washing).

Although sequence alignment algorithms that are well known in the art can be used for optimizing the sequence of a siRNA for use in the compositions and methods of the invention, the siRNA may also be identified or defined simply by assaying the ability of the siRNA to inhibit expression of a transgene in one or more assays described herein.

siRNAs can be produced in vivo or in vitro. Such RNAs can be synthesized enzymatically or by partial/total organic synthesis and any modified ribonucleotide can be introduced by in vitro enzymatic or organic synthesis. Endogenous RNA polymerase of the cell may mediate transcription of a siRNA in vivo, or cloned RNA polymerase can be used for transcription of the siRNA in vivo or in vitro. In one embodiment according to the invention, a siRNA used for attenuating an immune response to a protein encoded by a transgene by reducing transgene expression, is delivered using a vector. For example, a vector containing a transgene encoding a specific siRNA, may be administered to a host organism simultaneously or sequentially with the transgene whose expression is desired to be reduced or inhibited. For transcription from a transgene in vivo or transcription from an expression construct, a regulatory region (e.g., promoter, enhancer, silencer, splice donor and acceptor, polyadenylation) may be used to transcribe the RNA strand (or strands). siRNAs of the invention may be synthesized by a cellular RNA polymerase or a bacteriophage RNA polymerase (e.g., T3, T7, SP6). If synthesized chemically or by in vitro enzymatic synthesis, the siRNA may be purified prior to introduction into a cell or a host organism. For example, siRNA can be purified from a mixture by extraction, with a solvent or resin, precipitation, electrophoresis, chromatography, or a combination thereof. Alternatively, the siRNA may be used with no or a minimum amount of purification to avoid losses due to sample processing. The siRNA may be dried for storage or dissolved in an aqueous solution. The solution may contain buffers or salts to promote annealing, and/or stabilization of the duplex strands.

Various methods of administration of the siRNA as well as delivery of the transgene to a host cell or a host organism can be used.

The siRNA may be directly introduced into a cell or introduced into a cavity, interstitial space, or into the circulation of a mammal. The siRNA can also be introduced orally, or may be introduced by bathing an organism in a solution containing the siRNA. Methods for oral introduction of the siRNA include direct mixing of the siRNA with food for the organism, as well as engineered approaches in which a species that is used as food is engineered to express the siRNA and subsequently fed to the host organism desired to be treated. Physical methods of introducing nucleic acids include, for example, injection of the RNA directly into the cell or an organism. Vascular or extravascular circulation, the blood or lymph system, and the cerebrospinal fluid are some of the sites in a host organism where the siRNA may be introduced. Physical methods of introducing nucleic acids include injection of a solution containing the siRNA along with the gene therapy vector, bombardment by particles covered by the gene therapy vector and the siRNA, soaking the cell or organism in a solution of the siRNA, or electroporation of cell membranes in the presence of the gene therapy vector and siRNA. In some embodiments, the siRNA can be delivered to a specific organ, e.g., the liver, using hydrodynamic injection.

Other methods known in the art for introducing nucleic acids to cells may be used, such as lipid-mediated carrier transport, chemical-mediated transport, such as calcium phosphate, and the like.

The present invention also provides compositions comprising siRNA that can be administered to a mammal at the same time or after administration of a gene therapy vector comprising a transgene. In one embodiment, a siRNA composition of the invention is prepared as a pharmaceutical composition for administration to a host organism, for example, a human patient in need of gene therapy. Accordingly, a pharmaceutical composition comprising a siRNA, as used herein, may contain a pharmaceutically acceptable carrier to render the composition suitable for administration to a host organism.

A pharmaceutically acceptable carrier, as used herein, includes any or all solvents, dispersion media, coatings, antibacterial and antifungal agents, isotonic agents, absorption delaying agents and the like. It is understood that any conventional media or agent may be used so long as it is not incompatible with the compositions or methods of the invention.

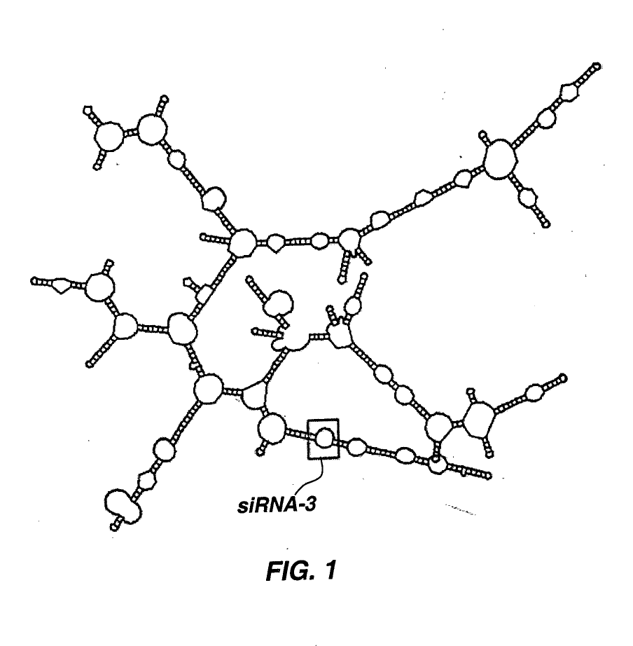

Design of siRNAs for use in the invention can be based on the two-dimensional structure of the mRNA expressed from a transgene targeted for inhibition. siRNA is typically complementary to a portion of the transgene which lies at least 50-100 nucleotides downstream of a transcription start site. A siRNA sequence typically includes anywhere between 21-25 nucleotides. For a 21 nucleotide long siRNA, it is desired that at least 10-11 nucleotides are G or C; however, a stretch of three or more G's is generally undesirable anywhere in the sequence. Additionally, a two nucleotide overhang at the 3′ end of the RNA is preferred. Available computer programs may be used for determination of a two-dimensional structure of an mRNA, e.g., the Genebee™ RNA secondary prediction program. Brodsky et al. (Genebee Molecular Biology Package for Biopolymer Structure Analysis, 1992). A two-dimensional structure for an mRNA typically includes several loop structures. An ideal loop structure used for designing a siRNA is on the periphery of the two-dimensional structure and includes at least 10 unpaired nucleotides on one side of the loop and at least 3 unpaired nucleotides on the other side of the loop. Additionally, a siRNA sequence can be searched against databases of known gene and protein sequences to ensure that it is specific to the transgene. The mRNA loop structure chosen for designing aGAL-siRNA-3 siRNA, which is used in the Examples is depicted in FIG. 1.

siRNA may be chemically synthesized using appropriately protected ribonucleoside phosphoramidites and a conventional DNA/RNA synthesizer. Each strand of the siRNA duplex can either be synthesized separately. siRNA can be duplexed in vitro before administration to a mammal or in vivo after each single strand is administered to a mammal. A siRNA duplex can also be synthesized as a stem-loop or hairpin structure composed of a strand and its complement. siRNA may also be readily obtained from a variety of commercial RNA suppliers, e.g., Dharmacon Research (Lafayette, Colo.), Pierce Chemical (Rockford, Ill.), Glen Research (Sterling, Va.), ChemGenes (Ashland, Mass.) and Xeragon, Inc. (Valencia, Calif.).

A siRNA can be assayed for inhibition of transgene expression in cells, for example, cells in culture, before it is administered to an immune competent host organism for attenuation of an immune response to the protein encoded by the transgene. For transfection of a siRNA into cells in culture, a transfection reagent such as Lipofectamine™ or Oligofectamine™ (Invitrogen, Carlsbad, Calif.) can be used. The transfection efficiency and the amount of reagent used will typically depend on the cell type as well as the degree of confluency of cells in culture and the number of times the cells have been passaged. Examples of cells that may be used for assaying inhibition of transgene expression, include, but are not limited to, CHO, HEK 293, NIH3T3, and HeLa.

In one example, the cell culture plates are divided into 4 different sets. To one set, a siRNA alone is added; to a second set, the siRNA plus the transgene to be inhibited is added; to a third set, the siRNA plus a control DNA or vector that does not carry the transgene is added; and to a forth set, only the DNA for transgene or a vector comprising the transgene is added. For transfection of siRNA or mixture of siRNA and DNA into cells, the siRNA alone or siRNA in combination with the transgene desired to be inhibited or siRNA in combination with the control DNA or the transgene DNA alone is mixed with cell culture medium in one tube. In another tube, the transfection reagent is mixed with the cell culture medium.

Each solution containing either the siRNA alone or siRNA in combination with a DNA, either the transgene or control DNA, or the transgene DNA alone, is mixed with the transfection reagent/cell culture medium mixture. The combination of the two solutions is mixed and incubated at room temperature for 20-25 minutes. The combination is subsequently added to the cells in culture. Each set of cells is harvested at different time points, for example, after 24, 36, 48, 72, 96, or 120 hours, subsequent to the addition of the siRNA or combination of siRNA and DNA to cells.

The effect of the siRNA on inhibition of transgene expression can be assayed either at the mRNA level or the protein level. For example, a siRNA specific for inhibition of a transgene will lead to a decrease or complete disappearance of the mRNA for transgene subsequent to transfection, however, it is expected that such an inhibition will be temporary and that the expression of the mRNA for the transgene will start to increase to the level expected in cells that are transfected with the transgene DNA alone, i.e., without siRNA.

The effect of a siRNA on expression of the transgene can also be assayed at the protein level, for example, by harvesting the transfected cells for total protein and detecting the amount of the protein encoded by the transgene using an antibody specific to the protein, using for example, Western blot analysis. Alternatively, the protein may be observed using a technique such as, immunofluorescence.

Therefore, siRNAs specific to a transgene or target gene desired to be inhibited can be designed using standard techniques known in the art and described herein, and such siRNAs can be tested for efficiency of inhibition in cell culture before they are administered to an immune competent host organism.

In further embodiments, the methods of this invention may be used in conjunction with enzyme replacement therapy (ERT) which entails direct infusion of a purified protein to a patient. ERT has been successfully used to treat lysosomal storage disorders, e.g., Gaucher's disease and Fabry disease, however, administration of purified enzyme often leads to an immune response to the replacement enzyme, reducing the effectiveness of the therapy. When the methods of the invention are used prior to ERT, the patient acquires immunologic tolerance to the replacement enzyme, thereby reducing or even eliminating an immune response when the enzyme is administered in ERT. The methods of inducing an immunologic tolerance to an immunogenic product comprise administering to the mammal a vector comprising a transgene encoding the immunogenic product and administering to the mammal a siRNA that temporarily inhibits transgene expression, wherein the siRNA is administered in an amount and for a period of time sufficient to reduce an immune response to the immunogenic product. It will be understood that for induction of tolerance the transgene product does not have to, but may be, expressed at therapeutic levels.

The methods of the invention allow administration of a therapeutic product which is otherwise immunogenic to a patient. The methods, for example, may include administration of a replacement enzyme, e.g., such as enzymes listed in Table 1. In some embodiments, the immunogenic product is an enzyme used in enzyme replacement therapy, e.g., α-Galactosidase A or glucocerebrosidase.

The methods of the invention are particularly useful in the treatment of diseases that are amenable to gene therapy, such as diabetes, hemophilia, Duchenne muscular dystrophy, familial hypercholesterolemia, cystic fibrosis, and lysosomal storage diseases.

In some embodiments, the invention provides methods and compositions for the treatment of a lysosomal storage disease such as Fabry disease and other diseases listed in Table 1. Fabry disease is an X-linked, recessive disorder resulting from a deficiency in α-galactosidase A. ERT involving administration of purified α-galactosidase A is currently being evaluated for treatment of patients; however, the purified enzyme usually has a short half-life in circulation and is rapidly cleared due to an immune response to the enzyme. See Ziegler et al., Hum. Gene Ther. 10: 1667-1682 (1999). This immune response is especially problematic in patients harboring a null mutation for the enzyme. However, the methods of this invention comprising administration of a transgene encoding α-galactosidase A and simultaneous or sequential administration of a siRNA that temporarily reduces or inhibits expression of the α-galactosidase A transgene result in a reduced immune response to α-galactosidase A when expression of the enzyme returns to therapeutic levels.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The examples do not in any way limit the invention.

### EXAMPLES

### Example 1

### siRNAs and Construction of Plasmids

siRNAs used in all experiments were obtained commercially from Xeragon, Inc. (Valencia, Calif.). The sequence of the control CAT-siRNA and aGAL-siRNA-3 (also referred to as siRNA-3) are depicted below:

| | |
|---|---|
| CAT-siRNA sense: | |
| GGAGUGAAUACCACGACGAUUUC | (SEQ ID NO:1) |
| CAT-siRNA antisense: | |
| AAUCGUCGUGGUAUUCACUCCAG | (SEQ ID NO:2) |
| aGAL-siRNA-3 sense: | |
| GUCUGAAGGUUGGAAGGAUGC | (SEQ ID NO:3) |
| aGAL-siRNA-3 antisense: | |
| AUCCUUCCAACCUUCAGACAC. | (SEQ ID NO:4) |

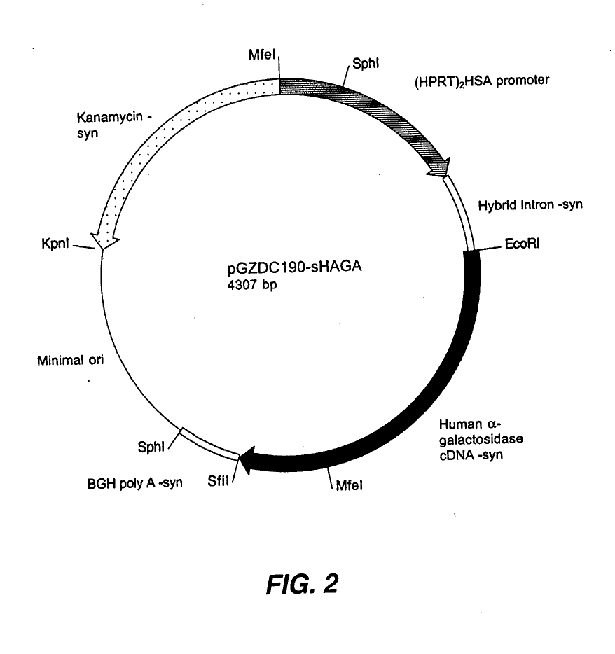

The pGZBDC190sHAGA plasmid was constructed as follows. The plasmid pSV2DC190HAGA, which contains two copies of the human prothrombin enhancer placed upstream of the human serum albumin promoter (hepatocyte-specific promoter), was digested with Cla1, the blunt ends were filled with Klenow polymerase and subsequently digested with Spel. The plasmid backbone of pGZB has been previously described by Yew et al. Mol. Therapy, 5(6): 731-8 (2002). Briefly, pGZB comprises a synthetic cytomegalovirus (CMV) immediate-early gene enhancer/promoter, a synthetic hybrid intron, a synthetic bovine growth hormone polyadenylation signal, a minimal replication origin region, and the synthetic kanamycin resistance gene. The plasmid pGZB was digested with Pmel and Xbal to remove the synthetic promoter, treated with Calf intestinal alkaline phosphatase (CIAP) and ligated to the blunt-ended Spel DC190 fragment to generate pGZDC190. The synthetic human α-galactosidase cDNA (sHAGA) fragment was isolated from the plasmid pGZBsHAGA using Sfil and EcoRI followed by blunt-end filling using Klenow polymerase. pGZDC190 was subsequently digested with Sfil and blunt-end filled with Klenow polymerase, treated with CIAP and ligated with the blunt-end sHAGA cDNA fragment to generate pGZBDC190sHAGA. A schematic representation of the vector, also referred to as pGZDC190sHAGA or pGZBDC190-shAGAL or pGZDC190-shaGAL or pDC190-agal, is shown in FIG. 2.

The features of plasmid pGZCUBIHAGA have been previously described in Yew et al., Mol. Therapy, 4(1): 75-82 (2001). This plasmid contains the cytomegalovirus (CMV) immediate-early gene enhancer, the human ubiquitin promoter, the human α-galactosidase A gene, a polyadenylation signal of the bovine growth hormone gene, a minimal replication origin region, and a synthetic kanamycin resistance gene.

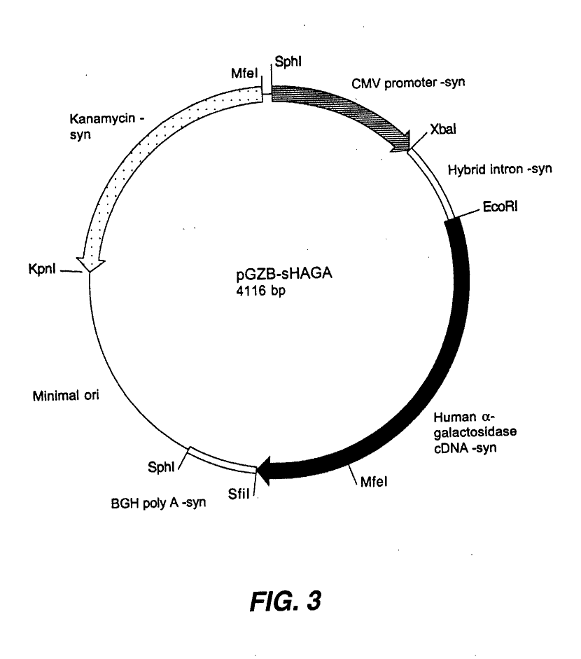

To create the pGZBsHAGA plasmid, a synthetic 1.3 kb human α-galactosidase A cDNA was assembled from oligonucleotides synthesized by Entelechon (Regensburg, Germany). The cDNA sequence was optimized for expression in human cells by removing rare codons and incorporating codons that are preferentially used in highly expressed human genes, for example, as described in Kim et al., Gene, 199: 293-301(1997). Additionally, all CpG dinucleotide sequences were eliminated to reduce immunogenicity of the plasmid. The ligation products received from Entelechon were cloned initially into the pCR2.1-TOPO plasmid (Invitrogen, Carlsbad, Calif.) to create plasmid pCR2.1TOPO-sHAGA. Site-directed mutagenesis was subsequently performed to correct errors in the sequence. The plasmid was digested with EcoRI and Sfil and the HAGA fragment was ligated into the EcoRI and Sfil cloning sites of pGZB, thereby resulting in plasmid pGZBsHAGA. A schematic representation of the pGZB-sHAGA vector, also referred to as pGZB-shaGAL, is shown in FIG. 3.

The plasmid pGZB-sSEAP was constructed as follows. A 1.6 kb synthetic cDNA fragment encoding the secreted form of human placental alkaline phosphatase (SEAP) was synthesized by Entelechon (Regensburg, Germany). The sequence was codon-optimized for expression in mammalian cells. In addition, all CpG dinucleotides were eliminated, but without altering the amino acid sequence. An EcoRI site was added to the 5′ end of the cDNA and a Sfil site was added to the 3′ end. The fragment was inserted into the EcoRI and Sfil sites of pGZB (Yew et al., Mol Ther. 5:731-8, 2002) to create pGZB-sSEAP.

### Example 2

### siRNA Reversibly Reduces Transgene Expression

Three groups BALB/c mice (Jackson Laboratory, Bar Harbor, Me.) were hydrodynamically injected with 2 ml of saline solution containing of the following: (1) 10 μg pGZDC190sHAGA; (2) 10 μg pGZDC190sHAGA plus 10 μg aGAL-siRNA-3; (3) 10 μg pGZDC190sHAGA plus 10 μg of control CAT-siRNA. The injections were performed over 7-120 seconds with a 27-gauge needle via a peripheral tail vein according to a hydrodynamic delivery protocol as described in Zhang et al., Human Gene Therapy, 10: 1735-37(1999)).

Plasma was collected by retro-orbital bleed for the measurement of human α-galactosidase protein (haGAL) by enzyme-linked immunosorbent assay (ELISA) as described in Ziegler et al., Hum. Gene Ther., 10:1667-82 (1999). Briefly, 96-microtiter plates (Corning, Corning, N.Y.) were coated with rabbit polyclonal anti-α-galactosidase antibody by incubating the plates with a solution containing the antibody (1.5 μg/ml) in 0.1M NaHCO₃, pH 9.5, for 1 hour at 37° C. The plates were blocked with 5% nonfat dry milk (Bio-Rad, Hercules, Calif.) in TBST (0.05 M Tris-HCl, 0.1M NaCl, 0.05% Tween 20, pH 7.5) at 4° C. for a minimum of 1 hour and then washed three times with ELISA plate wash buffer (300 μl/well; NEN Life Sciences, Boston, Mass.) using a model 1575 Immunowash® plate washer (Bio-Rad, Hercules, Calif.). Samples were diluted in 5% nonfat dry milk in TBST were loaded onto the plates and incubated for 1 hour at 37° C. and subsequently washed six times with ELISA buffer. Samples were incubated with biotinylated anti α-galactosidase antibody (1.25 μg/ml) (biotinylation was accomplished using the EZ-link-sulfo-NHS-LC biotinylated kit from Pierce, Rockford, Ill.) at 37° C. for 1 hour. After six additional washes with the ELISA plate wash buffer, the plates were incubated with 1 μg/ml streptavidin-horseradish peroxidase (HRP) (Pierce, Rockford, Ill.) at 37° C. for 30 minutes. The plates were subjected to six additional washes with the ELISA plate wash buffer and developed by incubating with a solution containing a 100 mg/ml concentration of 3,3′, 5,5′-tetramethyl benzidine dihydrochloride in substrate buffer (240 mM citric acid, 520 mM Na₂PO₄, pH 5.0) in a darkened room at room temperature for up to 30 minutes. The reactions were stopped by adding 100 μl of 2M H₂SO₄ to each well and the absorbance intensities at 450 nm were determined using a Bio-Rad model 450 plate reader. Concentrations were calculated from a standard curve generated using purified recombinant human α-galactosidase A (1000 μg/ml).

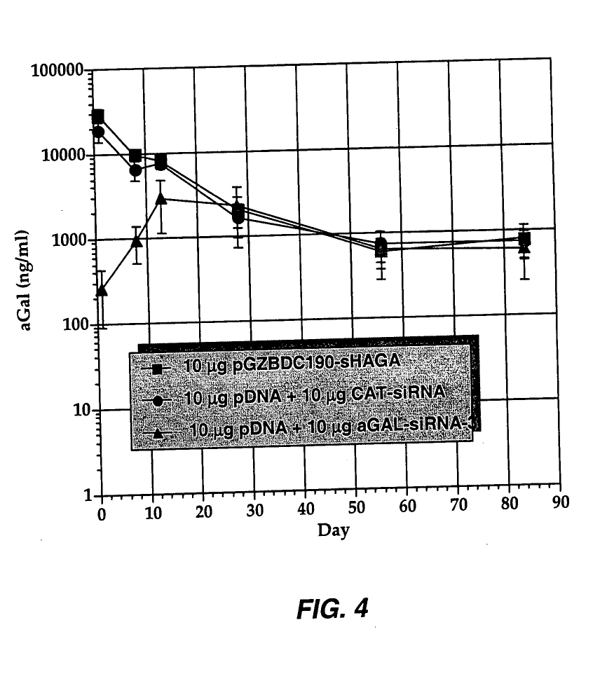

The expression level of the haGAL in mice was measured at various time points following injection of the pGZDC190sHAGA plasmid. Results of a representative experiment are depicted in FIG. 4. As shown in FIG. 4, the initial expression of haGAL from the pGZDC190sHAGA transgene vector was specifically reduced by co-administration of the haGAL-specific siRNA, aGAL-siRNA-3. Mice injected with pGZDC190sHAGA and aGAL-siRNA-3 showed a 100-fold reduction in haGAL plasma protein levels as compared to the mice injected with either the pGZDC190sHAGA alone or with pGZDC190sHAGA and control siRNA (CAT-siRNA). At day 21, the haGAL expression levels in mice treated with specific aGAL-siRNA-3, increased to a level comparable to that in control mice.

### Example 3

### siRNA Inhibits Transgene Expression in a Dose-Dependent Manner

BALB/c mice were hydrodynamically injected with 2 ml of saline solution containing of the following: (1) 4 μg pGZB-shaGal; (2) 4 μg pGZB-shaGal plus 5 μg CAT-siRNA; (3) 4 μg pGZB-shaGal plus 5 μg aGAL-siRNA-3, referred to as siRNA-3; or (4) 4 μg pGZB-shaGal plus 0.5 μg aGAL-siRNA-3. The plasma was collected at various time points and haGAL levels were measured by ELISA as described in Example 2. Additionally, anti-haGAL antibody titers in the were also measured at days 84, 99, and 112 post-injection using an assay described in Li et al., Mol. Therapy, 5 (6):745-754 (2002). Briefly, 96-well plates were coated with highly purified recombinant human α-galactosidase protein. Serial dilutions of the serum from mice at varying time points were added. Bound protein antibodies were detected using horseradish peroxidase (HRP)-conjugated goat anti-mouse IgG/IgM/IgA (Pierce, Rockford, Ill.). Titers were defined as the reciprocals of the highest dilution of serum that produced an OD₄₉₀ of less than or equal to 0.1.

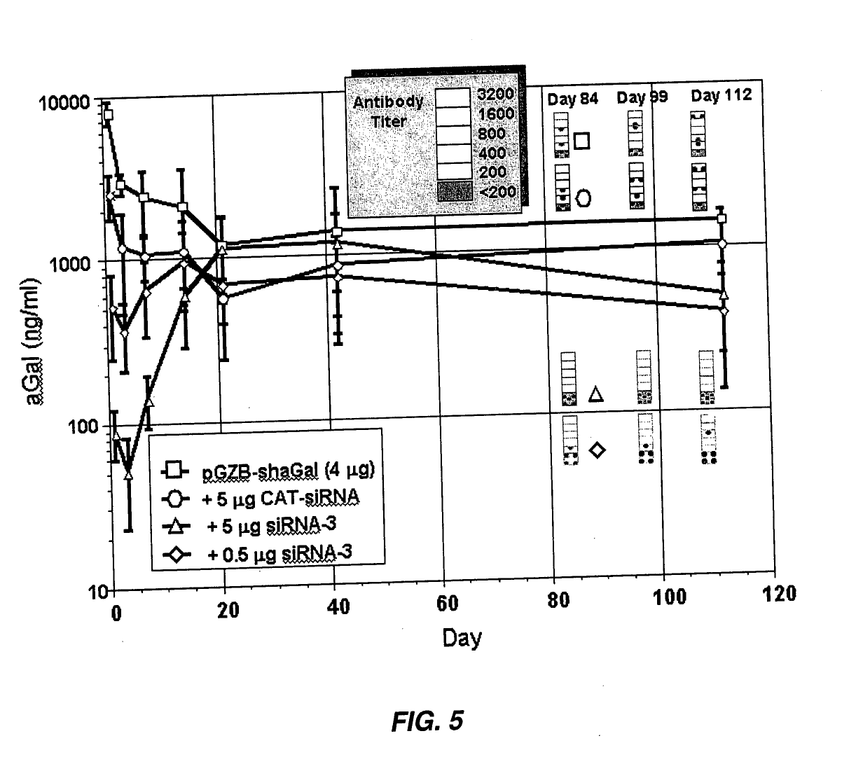

As shown in FIG. 5, the initial expression of haGAL was specifically reduced by co-administration of the haGAL-specific siRNA, aGAL-siRNA-3. The reduction in the haGAL expression levels was dose-dependent, e.g., mice that received 0.5 μg of aGAL-siRNA-3 exhibited a 10-fold reduction, whereas the 5 μg dose resulted in a 100-fold reduction.

### Example 4

### siRNA Reduces Specific Antibody Titer

Mice were treated as described in Example 3. Anti-haGAL antibody titers in were also measured at days 84, 99, and 112 post-injection using an assay described in Li et al., supra. Briefly, 96-well plates were coated with highly purified recombinant human α-galactosidase protein. Serial dilutions of sera from mice at varying time points were added. Bound protein antibodies were detected using horseradish peroxidase (HRP)-conjugated goat anti-mouse IgG/IgM/IgA antibody (Pierce, Rockford, Ill.). Titers were defined as the reciprocals of the highest dilution of serum that produced an OD₄₉₀ of less than or equal to 0.1.

FIG. 5 and Table 2 provide results of several experiments performed. These results demonstrate that administration of aGAL-siRNA-3 results in a sustained reduction of the anti-haGAL antibody titer as compared to control siRNA (CAT-siRNA) and no siRNA. This result was unexpected in view of the levels of haGAL expression (see Examples 2 and 3), i.e., the haGAL expression levels in mice treated with aGAL-siRNA-3 were comparable to the controls starting at around day 20. Mice that received a higher dose of aGAL-siRNA-3 (5 μg), had no significant anti-haGAL antibody titers at any time points evaluated. Only one out of five mice that received a lower dose of aGAL-siRNA-3 (i.e., 0.5 μg) developed a measurable but weak anti-haGAL antibody titer.

TABLE 2

| | pGZBsHAGA and | | | | pGZBsHAGA and | |
|---|---|---|---|---|---|---|
| | aGAL-siRNA | | pGZBsHAGA | | CAT-siRNA | |
| Antibody | # of | % of | # of | % of | # of | % of |
| Titer | mice | total | mice | total | mice | total |
| 3200 | 0 | 0 | 3 | 8.3 | 2 | 10.5 |
| 1600 | 1 | 2.2 | 3 | 8.3 | 2 | 10.5 |
| 800 | 4 | 8.7 | 5 | 13.9 | 3 | 15.8 |
| 400 | 3 | 6.5 | 5 | 13.9 | 4 | 21.1 |
| 200 | 5 | 10.9 | 6 | 16.7 | 3 | 5.8 |
| <200 | 33 | 71.7 | 14 | 38.9 | 5 | 26.3 |

### Example 6

### Expression of Transgene in α-galactosidase into Fabry Mice

Fabry (−/−) mice (Wang et al., Am. J. Human Genetics, 59: A208 (1996)) were bred at Genzyme Corp. (Framingham, Mass.) and allowed to mature to at least 4 months of age before use. Mice were hydrodynamically injected with 2 ml of saline solution containing of 10 μg pGZCUBIHAGA (non-tissue specific ubiquitin promoter). Organs were harvested from mice at varying time points. Briefly, the animals were perfused with phosphate buffered saline (PBS) prior to removing the organs, which were then frozen on dry ice and stored at −80° C. until ready for further processing. Blood was collected from the orbital venous plexus under anesthesia using heparinized microhematocrit capillary tubes at various times post-injection. Tissues were weighed, homogenized in the lysis buffer (27 mM citric acid, 46 mM sodium phosphate dibasic, 1% Triton X®100 and 1× protease inhibitor cocktail (Boehringer Mannheim, Indianapolis, Ind.), pH 4.6, and adjusted to a final concentration of 250 mg tissue per milliliter of the lysis buffer. The homogenized samples were subjected to three rapid freeze-thaw cycles and then stored at −80° C. For analysis, the frozen homogenates were first thawed and centrifuged at 10,000×g for 10 minutes at 4° C.

The α-galactosidase A levels were measured in the plasma, the liver, the spleen, the heart, the lung and the kidneys at days 1, 14, 28, and 42 post-injection. The amount of α-galactosidase A in the supernatants was determined as described in Ziegler et al., supra. Briefly, the amount of α-galactosidase A was determined using either 5 mM 4-methylumbelliferyl-α-D-galactopyranoside (4 MU-α-Gal) in the presence of 117 mM N-acetylgalactosamine at pH 4.4, or by ELISA, as described in Example 2.

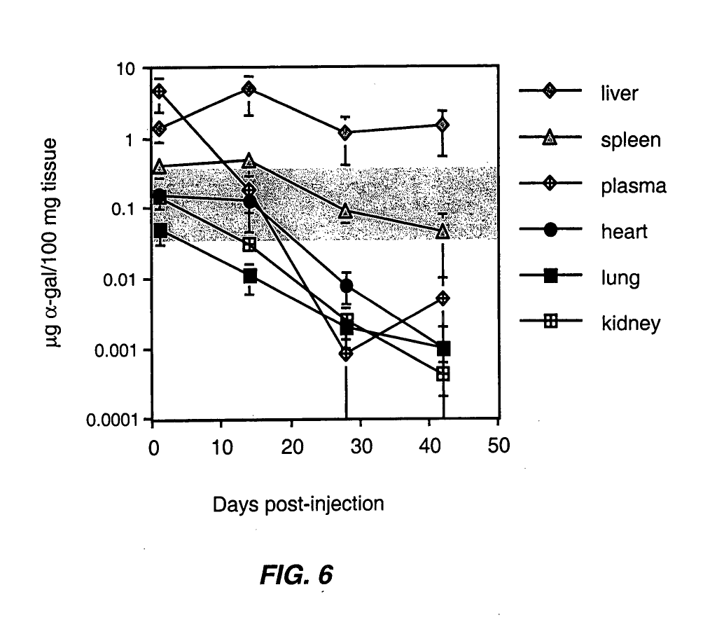

As depicted in FIG. 6, hydrodynamic delivery of plasmid expressing α-galactosidase led to the highest level of expression of the enzyme in the liver with lower levels of expression in all the other organs and in the plasma. Even at day 42, when the levels of expression declined in the other organs, expression in the liver remained high. These results suggest that the liver served as a depot organ following hydrodynamic injection.

### Example 7

### Transgene Product Alleviates Pathology in Fabry Mice

Fabry mice were treated and samples were collected as described in Example 6. Globotriaosylceramide (GL-3) accumulates in various organs of Fabry mice and also in humans suffering from Fabry disease. Levels of GL-3 in organs of Fabry mice were measured at days 1, 14, 28, and 42 following hydrodynamic delivery of pGZCUBIHAGA. Tissues were homogenized in chloroform-methanol (2:1 v/v) at a ratio of 0.1 ml/mg wet tissue. Samples (30 μl) were extracted with 0.6 ml of chloroform-methanol (2:1 v/v) by vortexing. After a 15 minute incubation at 37° C. on a rocking platform, samples were centrifuged to remove cell debris and one-fifth volume of water was added to the equivalent of 5 mg of tissue or to 25 μl of plasma. The phases were allowed to separate at 4° C. for 24 hours and then centrifuged to complete the separation. The lower phase (chloroform) was transferred to a clean glass tube and dried under nitrogen. To purify glycosphingolipids, the dried lipids were resuspended in 1 ml of chloroform and 0.5 mg equivalent applied to 500-mg Lichrolut RP-18 columns (EM Sciences, Gibbstown, N.J.). After washing with chloroform, the neutral glycosphingolipids were eluted from the columns with acetone-methanol (9:1 v/v), dried under nitrogen, and then resuspended in ethanol. Quantitation of GL-3 was performed using an ELISA as described in Ziegler et al. (supra) which relies on the affinity of GL-3 for the E. coli vertoxin B subunit (VTB) GL-3 was quantitated. Briefly, the lipids in ethanol (equivalent to 12.5 to 100 μg of tissue or 2.5 μl of plasma) were applied to a 96-well PolySorp™ plates (VWR Scientific Products, Bridgeport, N.J.) and dried to completion by incubation at 37° C. After blocking with 5% bovine serum albumin in Tris-buffered saline (TBS) for 1 hour at 37° C., the wells were reacted sequentially with VTB (400 ng/well), a monoclonal antibody against VTB (1 μg/well), and alkaline phosphatase-conjugated goat anti-mouse IgG antibody. Wells were developed with p-nitrophenylphosphate (1 mg/ml) in 10% diethanolamine, pH 9.6, at room temperature. The reactions were stopped with 100 μl of 5% EDTA and read at 405 nm in a Bio-Rad 450 plate reader. Standard curves were generated with porcine GL-3 (Matreya, Pleasant Gap, Pa.) using 5 to 100 ng/well.

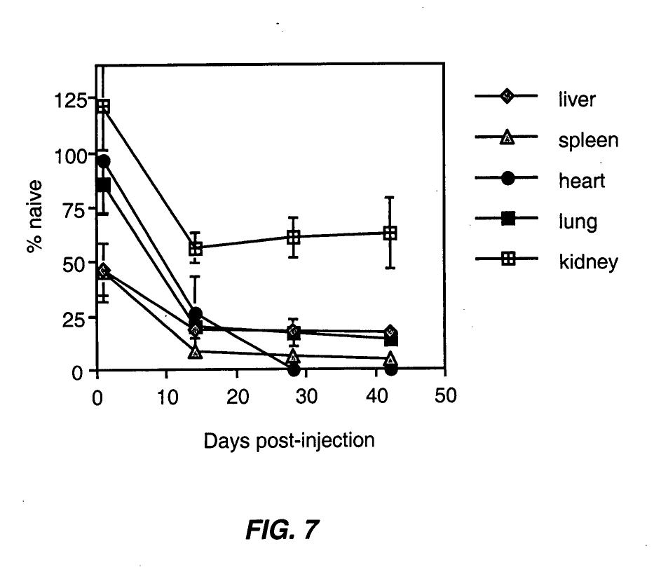

The level of GL-3 was measured at days 1, 14, 28, and 42 post-injection, as a percent of the GL-3 levels in corresponding organs of untreated mice (referred to as “% naïve”). As depicted in FIG. 7, the levels of GL-3 in mice that received pGZCUBIHAGA were high in all organs at day 1, and started to decline post-injection when measured at days 14, 28, and 42. These results indicate that that α-galactosidase A was effective in alleviating storage pathology in Fabry mice.

### Example 8

### Fabry Mice Generate Antibodies against α-galactosidase

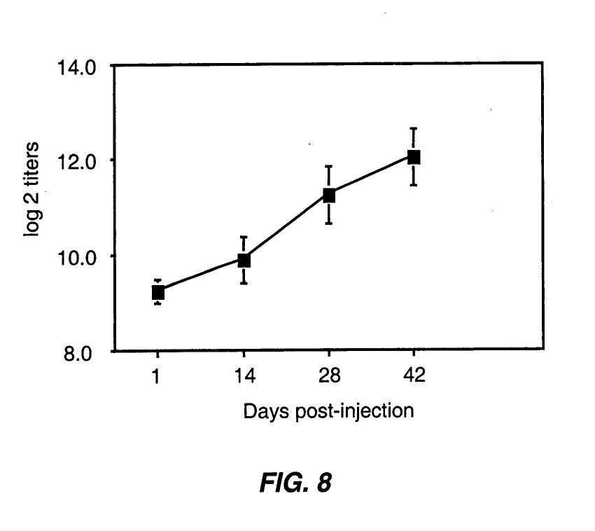

Female Fabry mice were hydrodynamically injected with 2 ml of saline containing 10 μg of pGZCUBIHAGA. Plasma was collected at days 1, 14, 28, and 42 post-injection and the anti-haGAL antibody titer was measured at days 1, 14, 28, and 42 post-injection, as described in Example 3. The results of a representative experiment are depicted in FIG. 8. As depicted in FIG. 8, Fabry mice generated anti-haGAL antibodies, and their titer increased consistently from day 1 to day 42.

### Example 9

### siRNA Reduces Specific Antibody Titer in Fabry Mice

Female Fabry (−/−) mice were hydrodynamically injected with 2 ml of saline solution containing: 10 μg pGZB-sHAGA; 10 μg pGZB-sHAGA plus 10 μg aGAL-siRNA-3; or 10 μg pGZB-sHAGA plus 10 μg CAT-siRNA. At days 1, 7, 14, 21, and 42, plasma was collected by retro-orbital bleed for measuring haGAL expression levels by ELISA, as described above in Example 2.

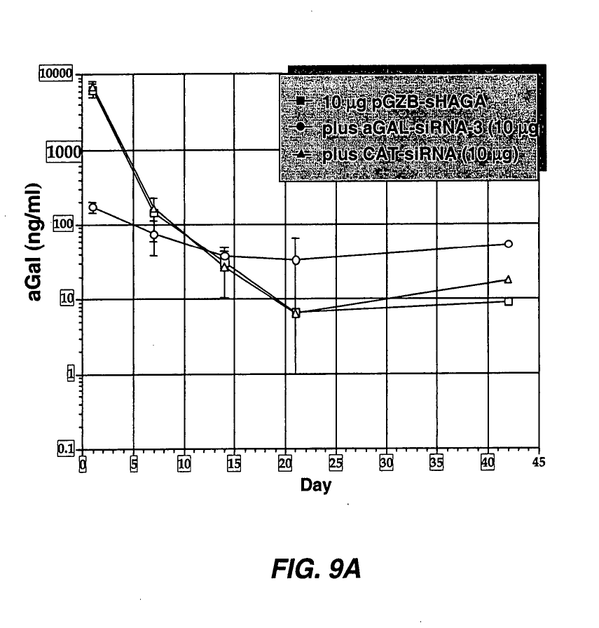

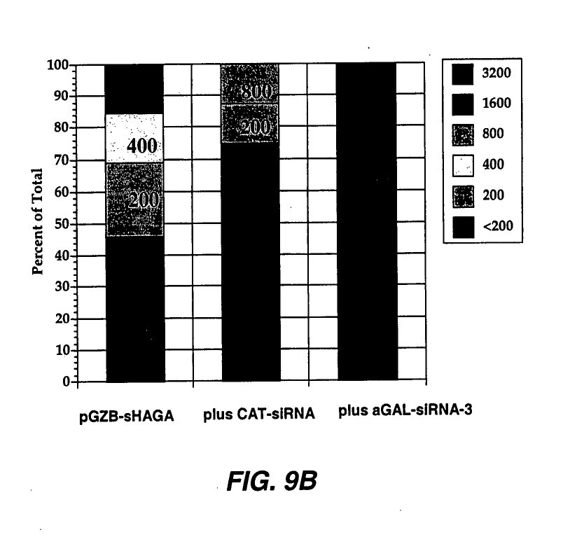

As shown in FIG. 9A, the initial expression of haGAL was specifically reduced by co-administration of haGAL-specific siRNA, aGAL-siRNA-3. In particular, a 200-fold excess of aGAL-siRNA-3 over pGZBsHAGA resulted in a 99% reduction in haGAL expression levels relative to the controls. The expression of haGAL returned to levels comparable to the controls in about three weeks. Additionally, as shown in FIG. 9B, co-administration of aGAL-siRNA-3 resulted in an overall reduction of the anti-haGAL antibody titers at day 42 as compared to no siRNA group or to the control siRNA (CAT-siRNA) group.

### Example 10

### siRNA Reduces Specific Antibody Titer in Fabry Mice

Fabry (−/−) mice were hydrodynamically injected with 2 ml saline containing: (1) 10 μg of pGZBDC190-shAGAL (tissue-specific promoter); (2) 10 μg of pGZBDC190-shAGAL plus 10 μg CAT-siRNA; (3) 10 μg pGZDC190-shaGAL plus 10 μg aGAL-siRNA-3. Plasma was collected over time and levels of haGAL expression were measured as described above in Example 2.

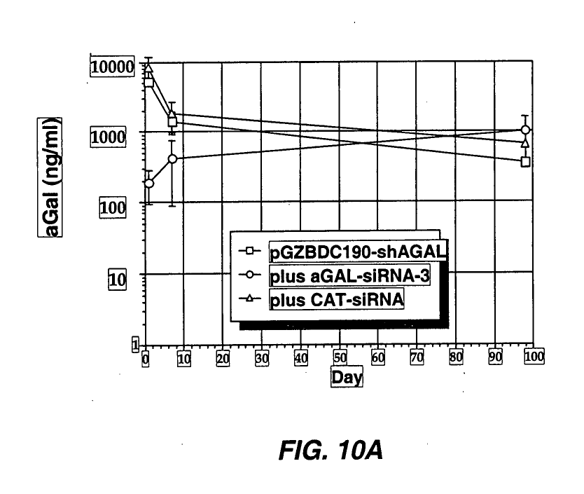

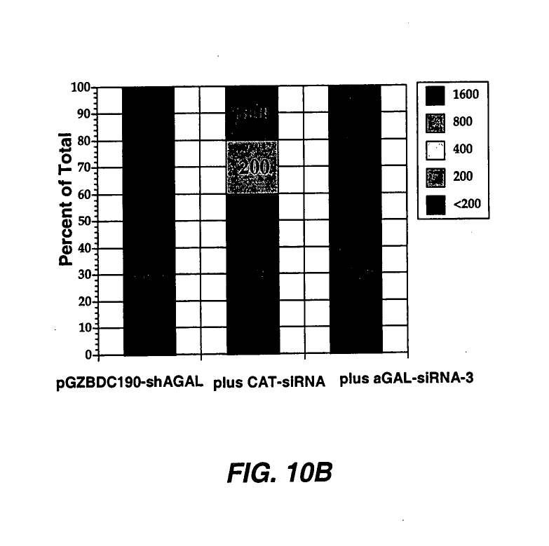

As shown in FIG. 10A, the initial expression of haGAL from the pGZBDC190-shAGAL vector was specifically reduced by co-administration of haGAL-specific siRNA, aGAL-siRNA-3. The expression of haGAL returned to levels comparable to the controls in about three weeks. Additionally, as shown in FIG. 10B, co-administration of aGAL-siRNA-3 resulted in an overall reduction of the anti-haGAL antibody titers at day 98 as compared to the control siRNA (CAT-siRNA).

### Example 11

### Induction of Tolerance to Fabrazyme in Fabry Mice

Fabry (−/−) mice were hydrodynamically injected with 2 ml saline containing: (1) 10 μg of pGZBDC190-shAGAL, referred to as pDC190-agal; (2) 10 μg of pGZBDC190-shAGAL plus 10 μg CAT-siRNA; (3) 10 μg pGZBDC190-shAGAL plus 10 μg aGAL-siRNA-3; or (4) 10 μg pGZB-sSEAP. Plasma was collected over time and levels of haGAL expression were measured as described above in Example 2.

Sixteen weeks after initial hydrodynamic injection, the Fabry mice were challenged with Fabrazyme (purified α-galactosidase A) to determine if immunologic tolerance to α-galactosidase had been achieved in any of the treatment groups. The challenge was performed by injecting the mice intraperitoneally with Fabrazyme in Complete Freund's Adjuvant (CFA). The groups were (1) no hydrodynamic injection at week 0 followed by Fabrazyme in CFA at week 16 [Naive+Fab]; (2) pGZBDC190-shaGAL injection followed by Fabrazyme in CFA at week 16 [pDC190+Fab]; (3) pGZBDC-190-shaGAL injection plus aGAL-siRNA-3 at week 0 followed by Fabrazyme in CFA at week 16 [pDC190/aGal-siRNA+Fab]; (4) pGZB-sSEAP injection followed by Fabrazyme in CFA at week 16 [pGZB-sSEAP+Fab]; (5) pGZB-sSEAP injection at week 0 followed by CFA only at week 16 [pGZB-sSEAP+CFA]; and (6) no hydrodynamic injection at week 0 followed by CFA only at week 16 [Naive+CFA].

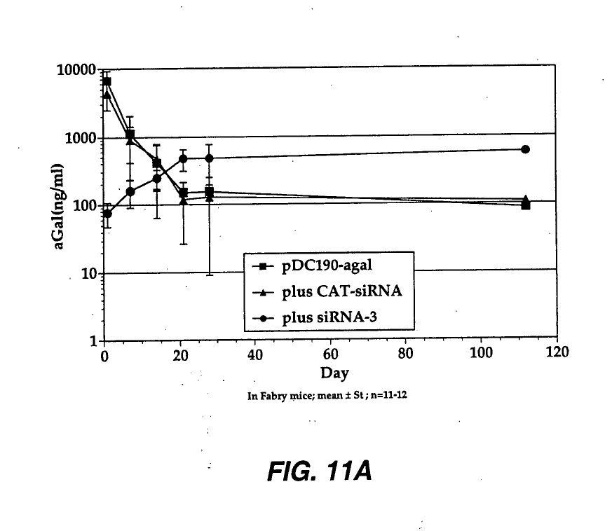

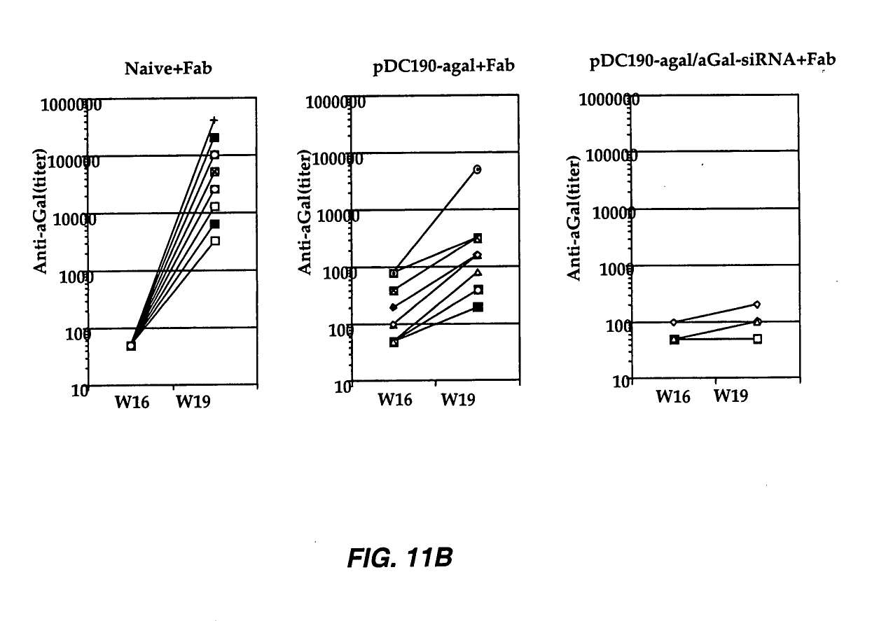

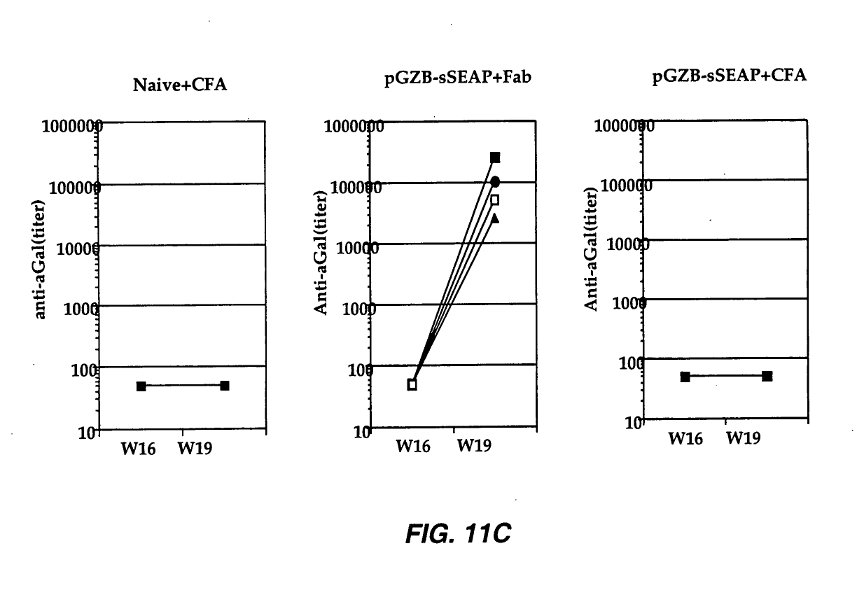

As demonstrated in FIG. 11A, the initial expression of haGAL from the pGZBDC190-shaGAL was specifically reduced by co-administration of haGAL-specific siRNA, aGAL-siRNA-3. In particular, co-administration of aGAL-siRNA-3 with pGZBDC190-shaGAL resulted in a 99% reduction in haGAL expression levels relative to the controls. The expression of haGAL returned to levels comparable to the controls in about three weeks.

As shown in FIGS. 11B at the week 16 time point (W16), this initial reduction in haGAL expression mediated a reduction in the anti-haGAL antibody titers in the pGZBDC190-shaGAL/aGAL-siRNA-3 treated mice [pDC190-agal/aGal-siRNA+Fab] as compared to mice that received either the control CAT-siRNA (data not shown) or no siRNA [pDC190-agal+Fab]. In addition, only mice treated with pGZBDC190-shaGAL plus aGAL-siRNA-3 [pDC190-agal/aGal-siRNA+Fab] failed to develop an appreciable anti-haGAL antibody titer three weeks (W19) after an intraperitoneal Fabrazyme challenge (see FIGS. 11B and 11C). Mice that 1) had received no plasmid at day 0 [Naive+Fab]; 2) had received only pGZBDC190-shaGAL in the absence of siRNA [pDC190+Fab] at day 0; or 3) had received only pGZB-sSEAP [pGZB-sSEAP+Fab] at day 0 all mounted a robust antibody response to the Fabrazyme challenge (see FIGS. 11B and 11C). Therefore, immunologic tolerance to Fabrazyme was generated by co-administration of an α-galactosidase encoding vector with an inhibitory siRNA that temporarily inhibited the α-galactosidase transgene expression.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method of reducing an immune response to a product of a transgene in a mammal comprising:
a) administering to a mammal a vector comprising a transgene encoding a product that is immunogenic in the mammal; and
b) administering to the mammal a small-interfering ribonucleic acid (siRNA) that temporarily inhibits transgene expression, wherein the siRNA is administered in an amount and for a period of time sufficient to reduce an immune response to the immunogenic transgene product when expressed at a therapeutic level.

2. The method of claim 1, wherein the vector is a gene therapy vector.

3. The method of claim 2, wherein the gene therapy vector is a plasmid DNA vector.

4. The method of claim 2, wherein the gene therapy vector is an adenoviral vector.

5. The method of claim 1, wherein the mammal is a human.

6. The method of claim 1, wherein the vector and the siRNA are administered simultaneously.

7. The method of claim 1, wherein the vector is administered prior to the siRNA.

8. The method of claim 1, wherein the siRNA is administered prior to the vector.

9. The method of claim 1, wherein the vector and the siRNA are administration by hydrodynamic delivery.

10. The method of claim 1, wherein the siRNA is at least 20 nucleotides in length.

11. The method of claim 1, wherein the siRNA is between 20-25 nucleotides in length.

12. The method of claim 1, wherein the siRNA is at least 25 nucleotides in length.

13. A method of treating or preventing a disease state in a patient comprising the steps of:
(a) administering to the patient a vector comprising a transgene encoding an immunogenic product that treats or prevents the disease state; and
(b) administering to the patient a small-interfering RNA (siRNA) that temporarily inhibits expression of the transgene, wherein the siRNA is administered in an amount and for a period of time sufficient to reduce an immune response to the product when expressed at a therapeutic level.

14. A method of treating a lysosomal storage disease in a mammal comprising:
(a) administering a vector comprising a transgene encoding an enzyme which is deficient or defective in the mammal with the lysosomal storage disease;
(b) administering to the mammal a siRNA that temporarily inhibits expression of the transgene encoding the enzyme in the mammal, wherein the siRNA is administered in an amount and for a period of time sufficient to reduce an immune response to the enzyme when it is expressed at therapeutic levels.

15. The method of claim 14, wherein the lysosomal storage disease is Fabry disease.

16. The method of claim 14, wherein the transgene encodes α-galactosidase protein.

17. The method of claim 14, wherein the siRNA comprises sequence of SEQ ID NO: 3 or a variant thereof which inhibits or reduces expression of α-galactosidase.

18. A method of reducing an immune response to an immunogenic product in a mammal comprising:
a) administering to the mammal a vector comprising a transgene encoding the immunogenic product; and
b) administering to the mammal a small-interfering ribonucleic acid (siRNA) that temporarily inhibits transgene expression, wherein the siRNA is administered in an amount and for a period of time sufficient to reduce an immune response to the immunogenic product.

19. The method according to claim 18, wherein the immunogenic product is an enzyme used in enzyme replacement therapy.


SEQUENCE LISTING (4 sequences)

SEQ ID NO: 1 - RNA, 23 nt, Artificial
  CAT-siRNA sense
ggagugaauaccacgacgauuuc

SEQ ID NO: 2 - RNA, 23 nt, Artificial
  CAT-siRNA antisense
aaucgucgugguauucacuccag

SEQ ID NO: 3 - RNA, 21 nt, Artificial
  aGAL-siRNA-3 sense
gucugaagguuggaaggaugc

SEQ ID NO: 4 - RNA, 21 nt, Artificial
  aGAL-siRNA-3 antisense
auccuuccaaccuucagacac